(19) United States
(12) Patent Application Publication
(10) Pub. No.: US 2022/0047311 A1
(43) Pub. Date: Feb. 17, 2022

(54) Surgical Method of Stabilizing Bone or Joint Fracture Using Flexible Line

(71) Applicant: Ahmad N. Ali, Avon, CT (US)

(72) Inventor: Ahmad N. Ali, Avon, CT (US)

(21) Appl. No.: 16/994,817

(22) Filed: Aug. 17, 2020

(51) Int. Cl. A61B 17/84; A61B 17/72; A61B 17/86

(52) U.S. Cl. CPC A61B 17/842; A61B 17/8645; A61B 17/7225

(57) ABSTRACT

A surgical method for stabilizing a fractured bone, comprises optionally realigning two portions of the bone impacted by the fracture, followed by drilling a hole through at least the first portion of bone and preferably the second portion of bone across the interface defined by the fracture. A flexible thread is threaded forward through the hole and a rear section of the thread anchored to the first portion of bone. The thread is pulled taut, holding the first bone portion relative to the second bone portion in a healing position. With the thread taut, a front section of the thread is anchored relative to the bone, thereby maintaining the first bone portion and second bone portion in the healing position.

### BACKGROUND

This disclosure relates generally to operative techniques for reduction of fractures in bones and/or joints, and more particularly to a method of fixing and stabilizing portions of a bone having suffered a fracture using a flexible line or thread.

Fracture reduction refers to the process of rigidly fixing portions of a fractured bone in a position relative to each other wherein proper healing can occur to avoid deformity, functional loss or re-fracture. It is a well-known technique for use in treating both human individuals and animals. When an individual or animal suffers a significant bone fracture, such as a displaced fracture, portions of the bone usually must be realigned and then fixed in place for a period of time while the bone heals. Depending on the location and type of the fracture, oftentimes the bone must be fixed with a plurality of different pieces of hardware, including one or more from the group consisting of pins, screws and plates, and many times with a combination thereof.

All of these known fixing techniques and hardware carry significant drawbacks. For example, drilling of screws into bone material causes large displacement of bone and makes the bone inherently more prone to another fracture. Implantation of hardware is especially problematic in sensitive areas of the body, such as areas that are close to articular surfaces.

Additionally, hardware is known to irritate and cause pain in the area of implantation and surrounding areas from pressure, rubbing and/or grinding. Implants are usually meant to remain in the patient's body indefinitely, but oftentimes pain or irritation is so severe that implants must be removed. Implant removal is usually a complex and delicate surgical procedure that necessarily yields a weakened bone structure due to the resulting empty space within the bone.

Thus, it would be useful to provide a surgical technique for fracture reduction that is less invasive to the individual or animal in need of treatment, and avoids or substantially reduces implantation of robust hardware into the body, which consequently reduces or avoids the drawbacks associated with known techniques for fracture reduction.

### BRIEF DESCRIPTION OF THE DRAWINGS

FIGS. 1A-2C depict prior art methods of stabilizing displaced femoral fractures in a canine patient using a plurality of pins and screws;

FIG. 3 is an X-ray image showing a displaced femoral fracture prior to reduction and stabilization;

FIGS. 4 and 5 show exemplary tools for use in practicing embodiments of the disclosed method;

FIG. 6A is an X-ray image of a canine patient in a ventrodorsal position with legs spread apart showing reduction of the femoral fracture from FIG. 3 using tools like those shown in FIGS. 4 and 5 according to the disclosed method;

FIG. 6B is another X-ray image of the canine patient in right lateral recumbent position showing reduction of the femoral fracture from FIG. 3 using the disclosed method;

FIG. 7 is an X-ray image of the canine patient from FIGS. 3, 6A and 6B approximately four months after reduction of the femoral fracture via the disclosed method, showing complete healing of the bone;

FIG. 8 shows an exemplary bone with a displaced fracture for which the disclosed fracture reduction method is effective for treating;

FIG. 9 shows the bone of FIG. 8 after a realignment step positioning the first and second bone portions relative to one another;

FIG. 10 shows the realigned bone of FIG. 9 after a step of boring a substantially linear hole through the first and second bone portions;

FIG. 11 shows the bone from FIG. 10 after a step of passing a thread through the hole; and

FIG. 12 shows the bone from FIG. 11 with the second end of the thread anchored to the second of the bone portions.

### DETAILED DESCRIPTION

Among the benefits and improvements disclosed herein, other objects and advantages of the disclosed embodiments will become apparent from the following wherein like numerals represent like parts throughout the several figures. Detailed embodiments of a method of surgically stabilizing a bone or joint fracture are disclosed; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrase “in some embodiments” as used herein does not necessarily refer to the same embodiment(s), though it may. The phrases “in another embodiment” and “in some other embodiments” as used herein do not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.

Further, the terms “substantial,” “substantially,” “similar,” “similarly,” “analogous,” “analogously,” “approximate,” “approximately,” and any combination thereof mean that differences between compared features or characteristics is less than 25% of the respective values/magnitudes in which the compared features or characteristics are measured and/or defined.

With reference to the drawings wherein like numerals represent like parts throughout the figures, a method of stabilizing bone fractures in an individual or animal, in particular displaced fractures, is shown and described. Most generally, the process utilizes the flexibility properties of a surgical thread to provide a force on portions of a fractured bone to urge the bone portions toward a substantially fixed healing position of the bone.

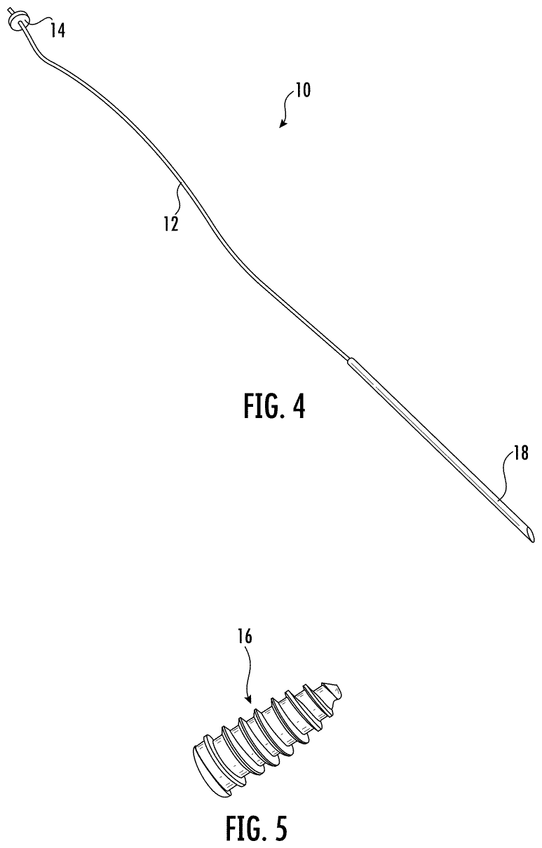

The disclosed process utilizes a surgical thread or similar line, such as that shown as reference numeral 12 in FIG. 4. As shown, the exemplary thread 12 carries a proximal stop 14 and may include or otherwise be attachable to a needle 18. As shown, the stop 14 comprises a thin disc carried on or near the proximal end of the thread 12 opposite the needle 18. Such thread products 12 are generally known in the surgical field and in use exclusively for joint stabilization techniques. The depicted thread 12 is exemplary in nature and non-limiting to the inventive concepts disclosed herein.

The disclosed process is particularly useful in treating displaced fractures that otherwise would require implantation of significant pieces of hardware, such as one or more of screws, pins and plates. In an exemplary embodiment, the disclosed process is appropriate for treating an individual or animal having suffered a fractured bone with the fracture separating the bone into at least two portions in need of being maintained in close proximity and alignment relative to one another for effective healing of the bone. With such bone fractures, proper reduction may require first resetting the alignment of the first and second bone portions relative to one another, followed by fixing the bone portions in the reset position. In the disclosed technique, after resetting the bone portions relative to each other, a hole 28 is drilled or otherwise bored through at least the first bone portion 22. Preferably, the hole is drilled through both of the first bone portion 22 and at least into the second bone portion 24, across the fracture line 26 that defines an interface between the first and second bone portions.

In cases of a comminuted fracture, a hole may be bored through multiple bone portions, but need not be bored through each separate portion of the bone. Further, a hole may be bored through only one portion of the bone and thereafter wrapped around other portions. Similarly, with a single fracture wherein one of the two portions is particularly thin and/or delicate, the hole may be bored through only one portion of the bone, i.e., the major portion of bone.

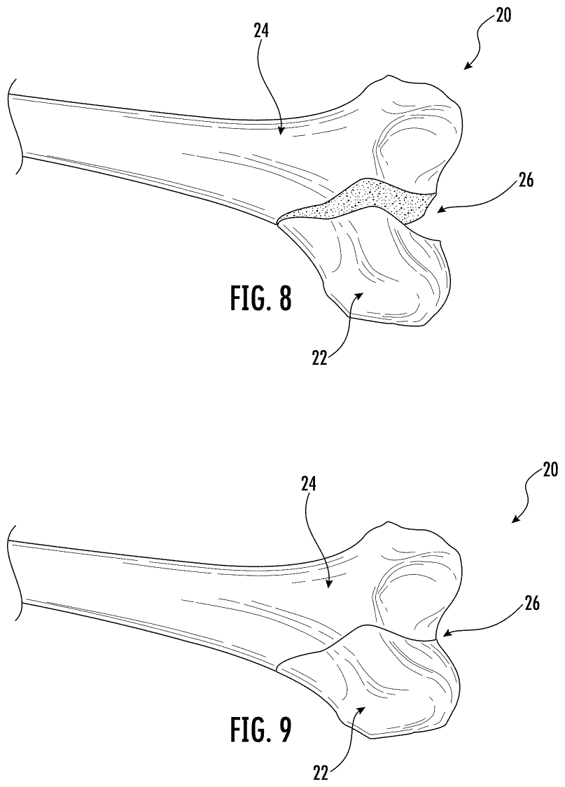

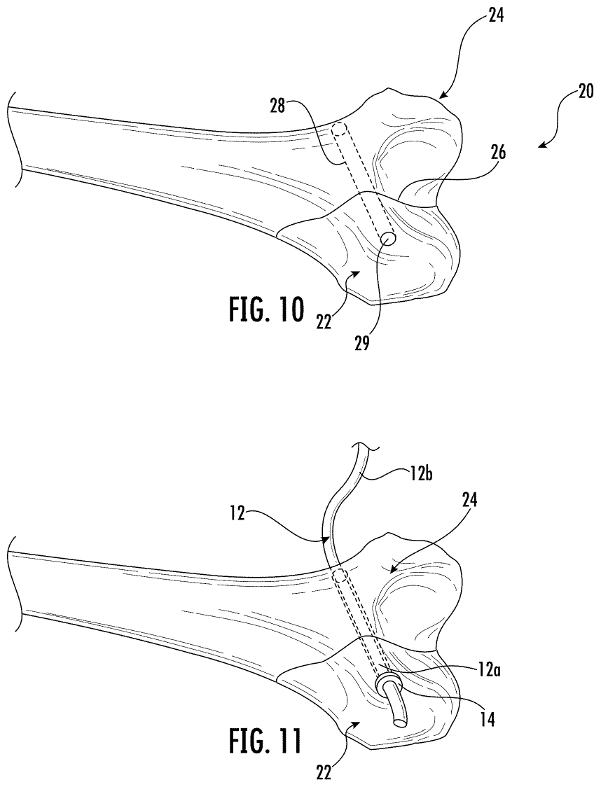

This embodiment of the disclosure will be described further with reference to a bone 20 with single displaced fracture, an example of which is depicted in FIG. 8. In cases of displaced fractures, such as that depicted, the first and second bone portions, 22 and 24, are initially realigned relative to one another to place the respective surfaces of the bone portions at the point of fracture in close proximity and/or contact (FIG. 9). Thereafter, a substantially linear hole 28 is bored through at least the first portion 22, and preferably through the second portion 24, across the fracture line 26 (FIG. 10). Preferably, the hole is bored from the first bone portion 22 that is smaller or closer to a sensitive area, such as an articular surface, to the second bone portion 24 that is more robust and/or further from a sensitive area.

Once the hole 28 (or holes) are bored, followed by removal of particulate and cleaning as known in the surgical arts, the thread 12 is inserted through the hole 28 from an inlet 29 at the outer surface of the first bone portion 22 and pulled forward through the hole until the stop 14 abuts the outer surface of the first bone portion 22, thereby anchoring the first end of the thread 12 thereto (FIG. 11). When the thread 12 is inserted with the stop against the first bone portion surface and pulled forward (distally), it is understood that the presence of the rear (proximal) stop 14 on the first end of the thread applies a substantially linear force on the first bone portion 22 that is parallel to the direction in which the thread 12 extends through the hole 28. This linear force holds the first bone portion 22 against the second bone portion 24 at the interface of the facture 26.

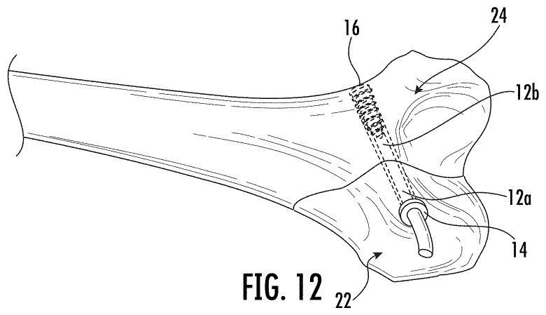

In the disclosed preferred embodiment of the method, the thread 12 is pulled taut and then anchored to the second bone portion 24 via an interference screw 16, such as that depicted in isolation in FIG. 5. Excess thread is thereafter cut, yielding a clean section of thread 12a within the hole 28 through the respective bone portions, 22 and 24 (FIG. 12). Other surgically-acceptable techniques for anchoring the thread in place may be employed, such as for example, self-ligation of the thread with non-slip knot sized larger than the hole. The integral linear force provided by the taut thread 12 extending between the first and second bone portions, anchored relative to the first portion 22 via the rear stop 14 and anchored to the second portion 24 via the interference screw 16, rigidly and permanently fixes the bone 20 in the proper reset alignment in a “healing position” with the respective opposed surfaces in close proximity and/or contact at the interface defined by the fracture 26. In the healing position, the respective portions of bone, 22 and 24, are maintained without movement relative to one another. In this manner, the fractured bone is maintained in the healing position without a need for invasive and cumbersome hardware as is currently known in the surgical arts. A single thread 12 extending linearly through the first bone portion 22 and second bone portion 24 across the fracture 26 and which is anchored to the respective bone portions proximate the first end and second end of the thread has shown remarkable strength and efficacy at reducing bone fractures in this manner.

In another embodiment, not depicted specifically, after the portions are realigned, a linear hole is bored through at least the first portion 22, and optionally the second portion 24. After removal of particulate, the thread 12 is pulled forward through the hole in at least the first bone portion 22 until the stop abuts the outer surface. A first (rear) section 12a of the thread remains within the hole in the bone as in the embodiment described above.

Unlike the embodiment described above, the remaining length of thread (i.e., second section of thread 12b) is wrapped tightly around a section of the outer surface of the second bone portion 24 in a manner wherein the thread 22 applies a compressive force on the second bone portion 24 that is generally transverse relative to the direction of extension of the wrapped second section of thread 12b. Optionally, the second section of thread 12b is additionally wrapped around the outer surface of the first bone portion. In this manner, the first portion of bone 22 and second portion of bone 24 are fixed in the healing position via the cooperative linear/parallel force of the first thread section 12a as applied by the stop 16 and the compressive forces provided by the tight wrapping of the second thread section 12b. Once the second section of thread 12a is wrapped appropriately to fix the bone portions in the healing position, the second end of the thread 12 may be anchored to a portion of the bone 20 with an interference screw 16 or otherwise ligated with application of a non-slip knot.

Importantly, in any embodiment, the inventive process is not limited in terms of the angle, direction or overall configuration of the thread 12, including the optional wrapping of the second section 12b of the thread. Consequently, the process is widely adaptable to virtually any bone and any fracture location and type. The flexible properties of the thread 12 provide another significant advantage over known fracture reduction processes that require rigid and invasive hardware at the location of the fracture—there is virtually no limitation on location for anchoring the second end of the thread 12 to the bone 20.

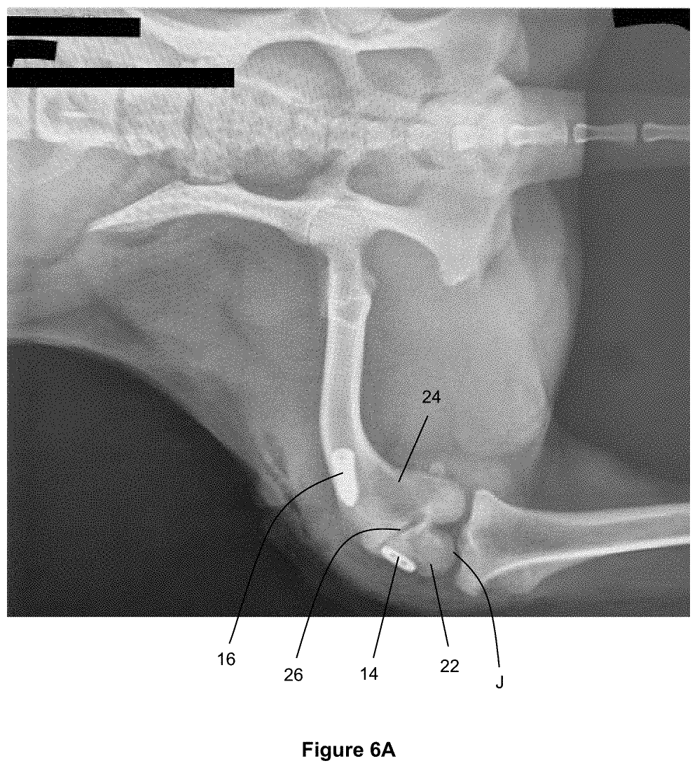

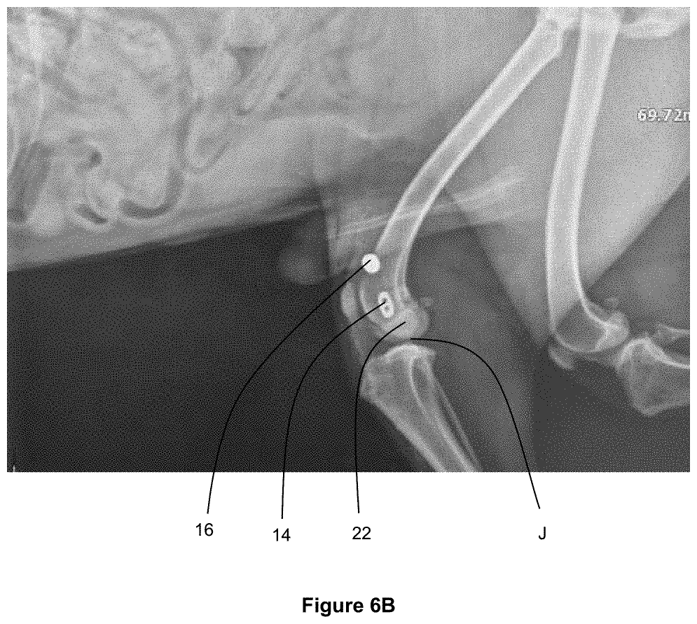

With further reference to the exemplary X-ray images of FIGS. 6A and 6B, in the case of the depicted distal femoral fracture, the second end of the thread is anchored at a position proximal of the fracture 26, and thus, positioned away from the sensitive area of the joint J and the fracture itself as well as in a particularly robust and durable area of the bone less susceptible to damage due to the anchoring process. The disc-shaped stop 14, which does not penetrate the bone 12 and has a very thin profile, may be positioned on the side of the bone closer to the sensitive articular surface without disruption of the joint or causing irritation.

The following Example 1 is illustrative of a single embodiment of the disclosed process, the inventiveness of which is not limited as such:

### EXAMPLE 1

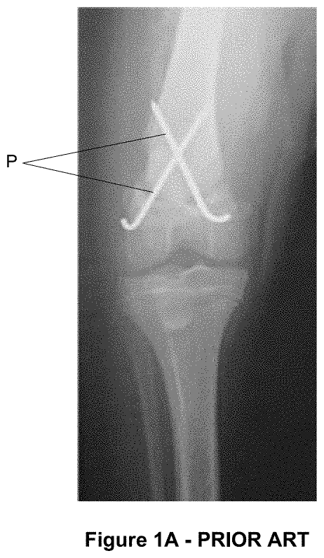

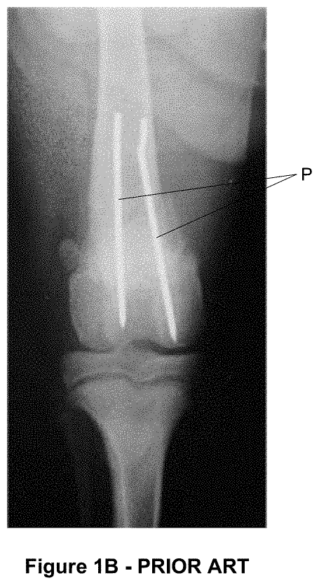

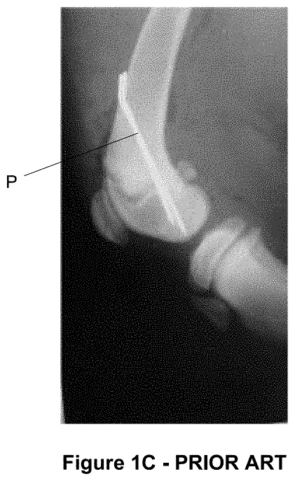

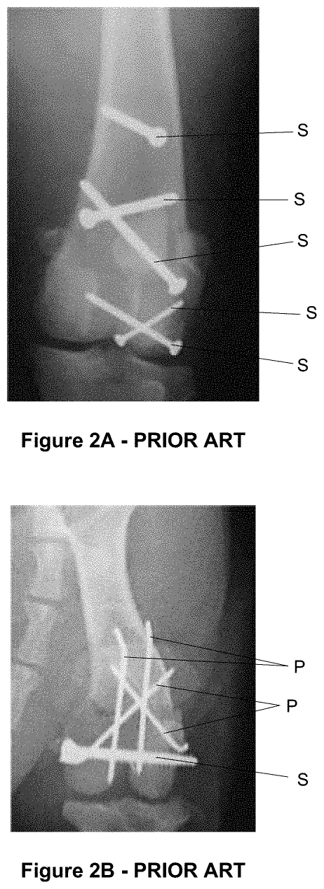

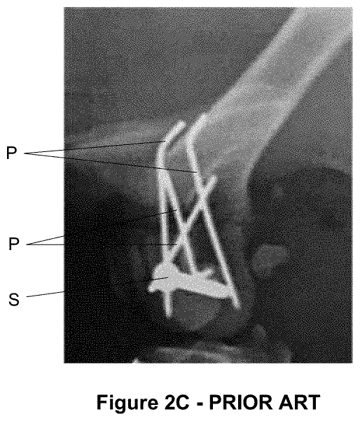

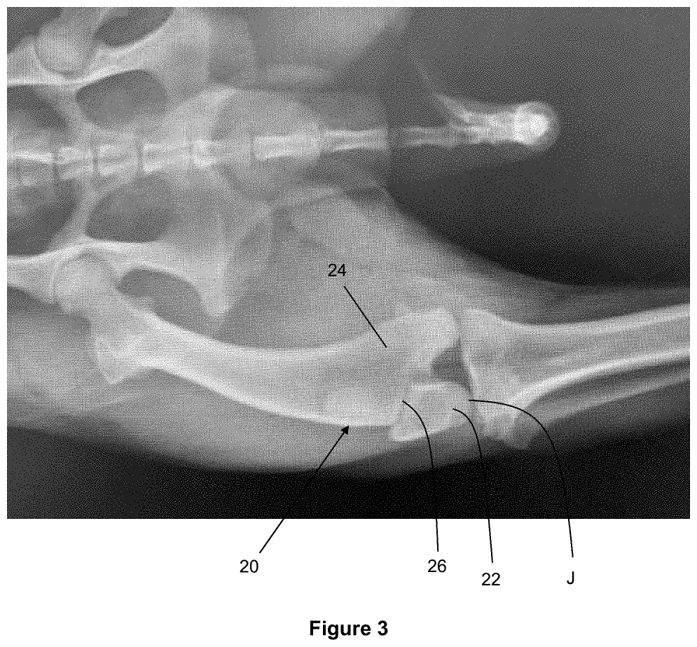

FIG. 3 is an X-ray image depicting a displaced fracture at a distal portion of the femur of a canine patient. As can be seen, the location of the fracture is in close proximity to the knee joint, which makes fracture reduction and further treatment risky and susceptible to irritation and possible further damage. Displaced fractures of the type shown in the X-ray image of FIG. 3 have always been reduced and stabilized using a plurality of large pins and/or screws, possibly in combination with plates to hold the portions of the fractured bone together at the interface defined by the fracture. Examples of prior art reduction techniques are shown in the X-ray images of FIGS. 1A-2C.

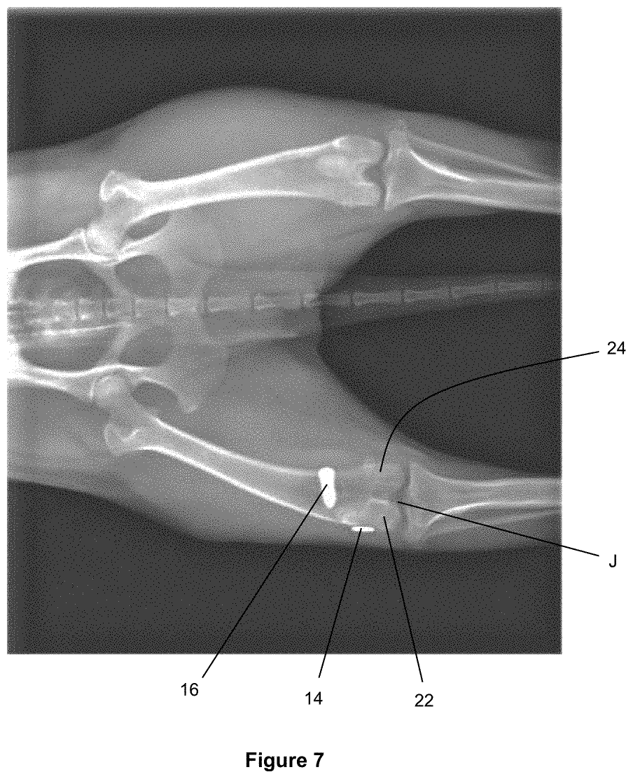

Instead of repositioning the bone portions and then implanting a series of hardware (pins, screws, plates), as would typically be done, the inventive reduction method was employed for treating the canine patient. After repositioning the portions of the bone in proper alignment relative to one another, a substantially linear hole was surgically drilled through the first portion 22 and the second portion 24 in a position proximal of the joint J, with the hole extending through the interface between the first and second portions at the fracture line. A flexible high strength thread 12 with a rear/proximal stop 14 was inserted at the inlet in the first bone portion and pulled forward through the hole in the first bone portion 22 and second bone portion 24 across the interface defined by the fracture 26 until the stop 14 was positioned in tight abutment with the outer surface of the first bone portion 22. The thread was pulled taut, exerting a force on the first bone portion 22 via the stop 14 urging the first bone portion 22 it toward the second bone portion 24 with both of the bone portions in their aligned healing position. In this manner, the linear force provided by the thread 12 and stop 14 is substantially aligned with the direction in which the thread is extended. Note that in the X-ray images of FIGS. 6A, 6B and 7, the thread 12 is not visible solely due to the limitations of X-ray technology.

With reference to FIGS. 6A and 6B, the thread 12 was fixed in the taut arrangement maintaining the first bone portion 22 and second bone portion 24 in the healing position via implantation of an interference screw 16 in the second bone portion 24. As such, a full and robust fracture reduction was accomplished via a single thread with stop and single interference screw rather than a series of invasive screws, pins and/or plates. As noted, the stop 14 is preferably a thin disc or similar shape with high strength and low thickness profile. The stop 14 provides a stark advantage in comparison to screws that necessarily include higher profile heads that often cause irritations to surrounding areas and the skin. Moreover, as the distal location of the femoral fracture is in close proximity to the knee joint J, the disclosed method avoids implantation of hardware near the sensitive articular surface. The interference screw 16 is the only piece of hardware embedded into the bone, and as shown, it is embedded at a location proximal of the fracture 24, removed from the articular surface and in a robust section of the bone 20. Optionally, the interference screw 16 can be omitted and the thread 12 fixed in a taut condition maintaining the bone portions aligned and healing position via a non-slip knot. Utilizing a non-slip knot in place of the interference screw 16 can be preferable in particularly sensitive locations wherein even the low profile implanted interference screw 16 can cause skin irritation or in particularly delicate bone portions.

In addition to the advantages noted above, the process of leveraging the bone portions in a healing position with a flexible thread 12 provides substantial adjustability with respect to the location of the anchoring screw 16, the only piece of implanted hardware. For example, the second thread end anchor position (i.e., location of the interference screw 16) can be adjusted to a location in the bone to minimize interference with sensitive areas like joints and delicate sections of bone. This phenomenon is shown clearly in FIGS. 6A, 6B and 7, wherein the screw 16 is implanted in a robust portion of the bone on the proximal side of the fracture, removed from the distal joint.

### Results

The patient with the reduced fracture shown in the X-ray images of FIGS. 6A and 6B was re-examined approximately two weeks following surgery. Follow-up X-ray images showed progress in healing of the fracture.

After approximately four weeks, the patient was running without noticeable pain or limp. Multiple follow-up videographs and X-rays show a completely healed fracture.

FIG. 7 is an X-ray image taken approximately four months after surgery. As shown, the fractured bone is completely healed in the proper alignment. The patient continues to show no signs of handicap or discomfort from the fracture, thereby indicating the efficacy of the disclosed method of treatment utilizing a flexible thread and anchoring it between two portions of bone impacted by the fracture to rigidly fix the portions together in a healing position.

Altogether, the disclosed method utilizing a flexible thread or line 12 to hold portions of a bone together for the purpose of fracture reduction has shown surprising efficacy, while reducing implantation of hardware, reducing discomfort to the patient and minimizing risk of further bone damage.

In a preferred embodiment, the flexible line 12 is formed of polyethylene fiber or a similar strong polymeric fiber with elastic properties. More preferably, the thread is formed of a thermoplastic, which may be selected from a polyethylene and a polyamide (i.e., nylon). More preferably, the thread is an ultra-high-molecular-weight polyethylene (UHMWPE), and even more preferably, the thread is a braided UHMWPE thread. In one preferred embodiment, the thread is an orthopedic suture manufactured and marketed by Intrauma S.p.A. for use in ligament repair (patella luxation and cranial cruciate ligament (CrCL) repair) surgical procedures, which are unlike the disclosed embodiments of a process for fracture reduction. The stop 14 is formed of a thin sheet of physiologically acceptable metal, such as titanium, for example. In another embodiment, the thread is replaced by a thin wire formed of titanium or similar metal.

Due to the flexible nature of the thread 12 and the adjustability of anchor locations provided, the inventive process provides virtually infinite options for leveraging portions of bone toward each another via anchoring, and optionally wrapping, allowing it to be employed in numerous locations for numerous different types of fractures and in delicate locations. For example, another specific surgical use is employing the process for reduction of a tibial tuberosity fracture, an injury that heretofore has been a particularly challenging condition to treat, and which has required implanting a significant amount of robust and invasive hardware.

Further, the disclosed process utilizing flexible thread for reduction of fractures can comprise a combination of the thread, as disclosed above, and an additional piece of hardware in a less invasive surgical assembly. For example, an embodiment exists that employs one or more rods acting as a support frame in combination with the anchored flexible thread 12. More specifically, one or more rods may be positioned on the outside of a broken bone, like a splint. A hole may be drilled in a strong portion of the bone with the thread extending through the hole and the first end anchored, as described above. The second section of thread may thereafter be wrapped around portions of the bone and the rods, as support, and then anchored to another portion of the bone.

While preferred embodiments of the foregoing have been set forth for purposes of illustration, the foregoing description should not be deemed a limitation of the invention herein. Accordingly, various modifications, adaptations and alternatives may occur to one skilled in the art without departing from the spirit and the scope of the present invention.

What is claimed is:

1. A method of treating an individual or animal having suffered a fractured bone, wherein the fractured bone comprises a first portion and a second portion with the fracture forming an interface between the first portion and second portion, the method comprising:
(a) boring a hole through at least the first portion and optionally the second portion;
(b) providing a flexible thread having a first end and a second end;
(c) passing the second end of the flexible thread through the hole;
(d) anchoring the first end of the flexible thread to the first portion of the fractured bone such that pulling of the thread through the hole applies a force on the first portion that is substantially parallel to the direction at which the thread extends through the hole; and
(e) anchoring the second end of the thread relative to the bone with a portion of the thread within the hole pulled taut, thereby fixing the first bone portion and second bone portion relative to one another in a healing position.

2. The method of claim 1, wherein
in step (a), the hole is bored through both the first portion and the second portion; and
in step (c), the second end of the flexible thread is passed through the hole in both the first portion and second portion.

3. The method of claim 2, wherein in step (e), the second end of the thread is anchored to the second portion of the bone.

4. The method of claim 3, wherein the second end of the thread is anchored to the second portion of the bone in a location that is not within the hole.

5. The method of claim 3, wherein the hole is substantially linear, and the taut thread extends substantially linearly through the hole in the first portion and second portion.

6. The method of claim 3, wherein the second end of the thread is anchored to the second portion of the bone via one of an interference screw and a non-slip knot with the thread pulled taut between the first portion of bone and the second portion of bone.

7. The method of claim 1, wherein the first end of the thread is anchored to the first portion of bone outside of the hole.

8. The method of claim 7, wherein the thread is anchored at the first end via a stop sized to abut with an outer surface of the first bone portion and configured to prevent the first end from passing through the first bone portion.

9. The method of claim 8, wherein the stop is a rigid substantially flat disc.

10. The method of claim 1, further comprising a step of:
wrapping a portion of the thread around the second portion of the fractured bone, thereby providing a force urging the second portion of the fractured bone toward the first portion of the fractured bone at the interface prior to step (e) of anchoring the second end of the thread.

11. The method of claim 1, wherein the fracture is a displaced fracture, comprising a step of realigning the first bone portion and second bone portion with one another in the healing position prior to step (a) of boring a hole.

12. The method of claim 1, wherein the taut thread contracts between its second end and first, whereby a force of contraction holds the first and second bone portions in the healing position.

13. The method of claim 12, wherein the taut thread only provides the force of contraction on the first and second bone portions.

14. The method of claim 1, wherein the thread is made of one or more thermoplastics.

15. The method of claim 14, wherein the thread is formed from ultra-high-molecular-weight polyethylene (UHMWPE) material.

16. A method of treating an individual or animal having suffered a fractured bone, wherein the fractured bone comprises a first portion and a second portion with the fracture forming an interface between the first portion and second portion, and wherein the first bone portion is displaced from the second bone portion, the method comprising:
(a) realigning the displaced first bone portion relative to the second bone portion such that the first bone portion meets the second bone portion at the interface, thereby defining a realigned position;
(b) boring a hole through the first portion and the second bone portion across the interface in the realigned position;
(c) providing a flexible thread extending between a first end and a second end;
(d) passing the second end of the flexible thread forward through the hole substantially linearly;
(d) anchoring the first end of the flexible thread to the first portion of the fractured bone such that pulling of the thread at the second end applies a force on the first portion forward toward the second bone portion;
(e) anchoring the thread relative to the second bone portion at a position forward of the first end with the thread pulled in a taut condition, thereby fixing the first bone portion and second bone portion relative to one another in the realigned position by a force of contraction provided by the taut thread; and
(f) allowing the first bone portion and second bone portion to heal together at the interface.

17. The method of claim 16, wherein the thread is anchored to the second bone portion via one of an interference screw and a non-slip knot with the thread pulled taut between the first bone portion and the second bone portion.

18. The method of claim 16, wherein the first end of the thread is anchored to the first portion of bone outside of the hole.

19. The method of claim 16, the thread pulled in the taut condition only provides the force of contraction on the first and second bone portions.

20. A method of treating an individual or animal having suffered a fractured bone, wherein the fractured bone comprises a first portion and a second portion with the fracture forming an interface between the first portion and second portion, the method comprising:
(a) aligning the first portion and second portion relative to one another at a desired healing position with the fracture forming an interface between the first portion and second portion;
(b) drilling an elongate hole from an outer section of the first portion through the first portion across the interface and through the second portion;
(c) providing a flexible thread extending from a proximal end to a distal end;
(d) threading the distal end of the flexible thread through the hole from the first portion and out from the second portion with a portion of the flexible thread extending through the hole;
(e) anchoring a proximal portion of the flexible thread relative to the first portion of the bone;
(f) pulling the flexible thread distally such that the portion of the thread within the first bone portion and second bone portion is taut, thereby forcing the first and second portions of bone toward each other at the interface; and
(g) anchoring the thread distal of the proximal portion to lock the thread taut extending within the elongate hole, thereby maintaining the first bone portion and second bone portion in position relative to one another to allow healing of the fractured bone.
